(19) United States
(12) Patent Application Publication
(10) Pub. No.: US 2006/0234987 A1
(43) Pub. Date: Oct. 19, 2006

(54) Neuroprotective benzo 1 3 oxathiol 2 ones

(76) Inventor: James B. Jaquith, QUEBEC (CA)

Correspondence Address: Philip A. Swain, Ph.D.;c/0 Aegera Therapeutics, 810 Chemin du Golf, Nun's Island, QC, H3E 1A8, CA

(21) Appl. No.: 10/564,941

(22) PCT Filed: Jul. 29, 2004
(86) PCT No.: PCT/CA04/01421

§ 371 Date: Jan. 18, 2006

Related U.S. Application Data
(60) Provisional application No. 60/490,528, filed on Jul. 29, 2003.

(51) Int. Cl. A61K 31/381; A61K 31/655; C07D 343/00

(52) U.S. Cl. 514/151; 549/33; 514/439

(57) ABSTRACT

This invention relates to benzo[1,3]oxathiol-2-ones, derivatives, and precursors thereof, and their use as neuroprotective agents in the treatment and prevention of neuronal disorders of the central and peripheral nervous systems.

### FIELD OF THE INVENTION

This invention relates to compounds and the use of compounds for the prevention of neuronal cell loss, for the treatment of nerve cell or axonal degradation, or for the induction of neurite regeneration, in either the central or peripheral nervous systems (CNS and PNS, respectively).

### BACKGROUND OF THE INVENTION

Neuronal damage can result from such diseases as Alzheimer's disease (AD), Huntington's disease (HD), Parkinson's disease (PD), amyotrophic lateral sclerosis (ALS), muscular dystrophy, multiple sclerosis (MS), diabetes, HIV, macular degeneration, retinal degeneration, from ischemic insults such as stroke in the brain, from retinal ganglion loss following acute ocular stroke or hypertension as in glaucoma, and from infection by viruses such as Hepatitis C and Herpes Simplex, and from the use of chemo-therapeutic agents used in the treatment of HIV and proliferative disease such as cancer.

Various neurotrophins such as Neuronal Growth Factor (NGF), brain derived growth factor (BDNF), neurotrophin-3 (NT-3), and others (NT-4, CNTF, GDNF, IGF-1), have been identified as key survival factors for neurons. NGF plays a critical role in the development and maintenance of cholinergic forebrain neurons of the CNS and neurons of the peripheral nervous system (PNS); neurons of the PNS are characterized as small fiber sensory neurons associated with pain and temperature sensation, in addition to neurons of the superior cervical ganglia and dorsal root ganglia (SCGs and DRGs, respectively). BDNF plays a role in motor neuron survival. Both BDNF and NT-3 are expressed in the CNS and serve similar purposes in multiple subsets of cortical and hyppocampal neurons; neurons of the CNS are characterized by those found in the brain, spinal chord, and eye. The removal of these and related trophic factors from in vitro cellular media results in the degradation of the axonal processes, leading to apoptosis of cultured neurons.

Localized tissue loss of NGF, or reduced axonal retrograde transport of NGF to the cell body, have been causally implicated in the development of peripheral neuropathies and neuropathic pain observed in diabetes and HIV patients. Several double blind Phase II clinical trials have found that the systemic administration of recombinant human NGF (rbNGF) (U.S. Pat. No. 5,604,202) displayed beneficial effects on neuropathic pain, physiology, and cognition related to these diseases (Apfel, S. C. et. al. JAMA, 248(17), 2215-2221; Apfel, S. C. Neurology 1998, 51, 695-702; McArthur, J. C. et al. Neurology 2000, 54, 1080-1088). Side effects related to rhNGF treatment included injection site pain, hyperalgesia, and other pain related symptoms. Despite these symptoms, a large number of patients continued rhNGF treatment after unblinding.

NGF binds to two cellular receptors: Trk A and p75. Binding of NGF to Trk A allows for the activation of Trk A via auto-phosphorylation. Activation (auto-phosphorylation) of Trk A results in the recruitment of components of intracellular signaling cascades and the initiation of processes resulting in neuronal survival and growth. NGF binding to p75, in the absence of Trk A, initiates a pro-apoptotic cascade, ultimately leading to cell death via activation of the p75/JNK pathway. SHP1 is a phosphatase which selectively dephosphorylates Trk A interrupting both the pro-survival and pro-growth signals in neurons (Kaplan, D. R.; Miller, F. D. Current Opinion in Neurobiology 2000, 10, 381-391). Compounds which augment the NGF signaling process or selective inhibitors of SHP 1 represent novel approaches to promoting neuronal survival growth and repair.

Various chemotherapeutic drugs such as Taxol™, cisplatin, vinblastine, and vincristine, cause dose dependent peripheral neuropathies, characterized by peripheral pain and loss of function. In many cases these neuropathies effectively limit the amount, and duration, of chemotherapy given to patients. For example, upwards of 50% of patients receiving Taxol™ chemotherapy experience severe and/or cumulative peripheral neuropathies. The progression of the neuropathy necessitates the use of various dosing regimes to reduce the incidence of neuropathy. Regression of the neuropathy is often observed between treatment cycles and following the final treatment. The degree and duration of recovery varies largely between patients. In addition to peripheral neuropathies, cisplatin treatment may result in auditory loss, especially in children, with minimal recovery after completion of treatment.

In order to identify compounds which mimic the positive effects of NGF on peripheral neurons, but which lack the inherent difficulties associated with the use of recombinant human proteins and the rhNGF related hyperalgesia, several in vitro screens have been developed using a variety of neurotoxic insults. PNS neurons such as the superior cervical ganglion (SCG) and dorsal root ganglion (DRG) undergo apoptosis when subjected to NGF withdrawal. Treatment with chemotherapeutic agents such as Taxol™, cisplatin, vinblastine, vincristine and selected anti-viral agents also may induce neuronal apoptosis. Similarly, neurons of the CNS, such as cortical neurons, are sensitive to various neurotoxic agents such as beta-amyloid, NMDA, osmotic shock, Taxol™ and cisplatin.

It is, therefore, desirable to provide compounds which protect neurons from neurotoxic insults such as those mentioned above.

Examples of benzo[1,3]oxathiol-2-ones have been previously described. Their uses include fungicides (U.S. Pat. No. 4,349,685) and herbicides (Webb, S. R., et al., J. Agric. Food Chem., 2000, 48, 1219). A series of 2-(aryloxymethyl)piperidine azacyclic nicotinic acetylcholine receptor ligands were prepared (Elliot, R. L., et al. Bioorg. Med. Chem. Lett., 1996, 6, 2283). One compound displays the benzo[1,3]oxathiol-2-one moiety; however, this pendent moiety appears to have little to do with the activity of this class of compounds.

There are several synthetic routes to benzo[1,3]oxathiol-2-ones which may be found in the above references (also see U.S. Pat. No. 2,332,418). Also included are methods for the functionalization of benzo[1,3]oxathiol-2-ones.

### SUMMARY OF THE INVENTION

Applicants have made the new and unexpected discovery that novel benzo[1,3]oxathiol-2-ones are useful as neuroprotective agents. Benzo[1,3]oxathiol-2-ones and the use of benzo[1,3]oxathiol-2-ones for the prevention of neuronal cell loss or for the treatment of nerve cell or axonal degradation in either the central or peripheral nervous systems (CNS and PNS, respectively), or for the induction of neurite regeneration is disclosed.

Compounds that prevent loss of, maintain, or rescue neurons would be useful in the treatment of the peripheral neuropathies observed in diseases such as diabetes, and HIV. Compounds which protect neurons from chemotherapeutic toxicity, if given concurrently with, or following chemotherapeutic treatment will allow for the use of increasing concentrations of chemotherapeutics and/or extend the duration of chemotherapy treatments. Alternatively, enhanced recovery will be observed if such compounds are given during the recovery stages, and post treatment. These compounds will also be useful in the treatment of neurodegenerative diseases of the CNS, such as AD, PD, HD, stroke, MS, amyotrophic lateral sclerosis (ALS), macular degeneration, glaucoma, optical stroke, retinal degeneration, and the like.

Selected analogs of benzo[1,3]oxathiol-2-ones can be used to prevent or alleviate such neuronal damage, and can be used for the treatment of neurodegenerative diseases of the CNS and/or PNS, for the inhibition of selected phosphatases, for inhibiting the degradation, dysfunction, or loss of neurons of the CNS and/or PNS, for enhancing the phenotype of neuronal cell types, and for preserving the axonal function of neuronal and synaptic processes of the CNS and/or of the PNS, and for the induction of neurite regeneration. This invention also relates to methods for the preparation of these compounds.

Accordingly, in the first embodiment of the present invention, there is provided compounds represented by Formula I: 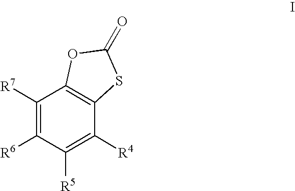
or pharmaceutically acceptable salts thereof wherein:

R⁴, R⁵, R⁶, and R⁷ are independently selected from the group consisting of:

H, halogen, cyano, azide, formyl, substituted and unsubstituted C(1-8) alkyl, C(1-8)fluoroalkyl, substituted and unsubstituted aralkyl, substituted and unsubstituted aryl, substituted and unsubstituted heteroaryl, substituted and unsubstituted biphenyl,

XR⁸, wherein X is S or O, and R⁸ is selected from the group consisting of H, substituted and unsubstituted C(1-8)alkyl, C(1-8)fluoroalkyl, substituted and unsubstituted acyl, substituted and unsubstituted arylcarbonyl, substituted and unsubstituted heteroarylcarbonyl, substituted and unsubstituted alkylaminocarbonyl, substituted and unsubstituted arylaminocarbonyl, substituted and unsubstituted heteroarylaminocarbonyl, substituted and unsubstituted aralkyl substituted and unsubstituted aryl, substituted and unsubstituted heteroaryl, substituted and unsubstituted alkylsulfonyl, substituted and unsubstituted arylsulfonyl, substituted and unsubstituted heteroarylsulfonyl and

NR⁹R¹⁰, wherein R⁹ and R¹⁰ are independently selected from the group consisting of H, substituted and unsubstituted C(1-8) alkyl, C(1-8) fluoroalkyl, substituted and unsubstituted acyl, substituted and unsubstituted arylcarbonyl, substituted and unsubstituted heteroarylcarbonyl, substituted and unsubstituted alkylaminocarbonyl, substituted and unsubstituted arylaminocarbonyl, substituted and unsubstituted heteroarylaminocarbonyl, substituted and unsubstituted aralkyl substituted and unsubstituted aryl, substituted and unsubstituted heteroaryl, substituted and unsubstituted alkylsulfonyl, substituted and unsubstituted arylsulfonyl, substituted and unsubstituted heteroarylsulfonyl, or wherein R⁹ and R¹⁰ are combined to form a heteroalkyl, substituted heteroalkyl, heteroaryl, and substituted heteroaryl ring system; and wherein

R⁴ and R⁵ may be combined to form a cycloalkyl, substituted cycloalkyl, heterocycloalkyl, substituted heterocycloalkyl, aryl, substituted aryl, heteroaryl, or substituted heteroaryl ring system; and

R⁶ and R⁷ may be combined to form a cycloalkyl, substituted cycloalkyl, heterocycloalkyl, substituted heterocycloalkyl, aryl, substituted aryl, heteroaryl, or substituted heteroaryl ring system.

In one aspect, compounds of this invention are those compounds represented by Formula I which display bulky substituents such as naphthylthio, haloaryl, or biphenyl at R⁷, and a hydroxyl, a substituted acetoxy or alkylaminocarbonyl moiety containing an amino, mono- or disubstituted amino, pyridyl, or piperidinyl moiety, substituted or unsubstituted arylcarbamoyloxy, or substituted or unsubstituted heteroarylcarbamoyloxy moiety at R⁵.

In another aspect, compounds of the invention are those compounds represented by Formula I, wherein R⁷ is selected from the group consisting of substituted or unsubstituted arylthio, substituted or unsubstituted heteroarylthio, and R⁵ is selected from the group consisting of hydroxyl, substituted alkylcarbonyloxy or substituted alkylaminocarbonyloxy moiety containing an amino, mono- or disubstituted amino, pyridyl, piperidinyl, piperazinyl, morpholino, thiomorpholino, or pyrrolidinyl moiety, substituted or unsubstituted prolinyloxy, substituted or unsubstituted heteroarylcarbonyloxy, substituted or unsubstituted heteroarylaminocarbonyloxy.

In another aspect, compounds of the invention are those compounds represented by Formula I, wherein R⁷ is substituted or unsubstituted haloaryl, and R⁵ is selected from the group consisting of hydroxyl, substituted alkylcarbonyloxy or substituted alkylaminocarbonyloxy moiety containing an amino, mono- or disubstituted amino, pyridyl, piperidinyl, piperazinyl, morpholino, thiomorpholino, or pyrrolidinyl moiety, substituted or unsubstituted prolinyloxy, substituted or unsubstituted heteroarylcarbonyloxy, substituted or unsubstituted heteroarylaminocarbonyloxy.

In another aspect, compounds of the invention are those compounds represented by Formula I, wherein R⁵ is a substituted alkylcarbonyloxy or substituted alkylaminocarbonyloxy moiety containing an amino, mono- or disubstituted amino, pyridyl, or piperidinyl, piperazinyl, morpholino, or pyrrolidinyl moiety, substituted or unsubstituted heteroarylcarbonyloxy, substituted or unsubstituted heteroarylaminocarbonyloxy, substituted or unsubstituted prolinyloxy, and R⁷ is a substituted or non-substituted biphenyl moiety.

In another aspect, compounds of the invention are those compounds represented by Formula I, wherein R⁴ and R⁶ are hydrodgen.

In one aspect, the compound may be: 5-hydroxy-7-((2-naphthyl)sulfanyl)benzo[1,3]oxathiol-2-one (23), 5-(N-Butylcarbamoyloxy)-7-((2-naphthyl)sulfanyl)benzo[1,3]oxathiol-2-one (25), 5-(N-(4-methoxyphenyl)carbamoyloxy)-7-((2-naphthyl)sulfanyl)benzo[1,3]oxathiol-2-one (26), 7-(2-chlorophenyl)-5-hydroxybenzo[1,3]oxathiol-2-one (36), 7-(2,4-dichlorophenyl)-5-hydroxybenzo[1,3]oxathiol-2-one (41), 7-(2,5-dichlorophenyl)-hydroxybenzo[1,3]oxathiol-2-one (42), 7-(3,4-dichlorophenyl)-5-hydroxybenzo[1,3]oxathiol-2-one (43), 7-(4-bromophenyl)-5-hydroxybenzo[1,3]oxathiol-2-one (44), 5-hydroxy-7-(3-iodophenyl)benzo[1,3]oxathiol-2-one (45), 6-(2,6-dimethylphenyl)-5-hydroxybenzo[1,3]oxathiol-2-one (50), 7-biphenyl-5-hydroxybenzo[1,3]oxathiol-2-one (54), 5-(3-pyridylcarbonyloxy)-7-biphenylbenzo[1,3]oxathiol-2-one (55), or 5-(N,N, dimethylaminomethylcarbonyloxy)-7-biphenylbenzo[1,3]oxathiol-2-one (61).

In one aspect, the invention provides a pharmaceutical composition for the prevention of neuronal cell loss or for the treatment of nerve cell or axonal degradation, comprising the compound of Formula I as described herein, together with a suitable pharmaceutically acceptable diluent or carrier.

In another aspect, the invention relates to the use of compounds of Formula I described herein, for the prevention of neuronal cell loss or for the treatment of nerve cell or axonal degradation or for the manufacture of a medicament for the prevention of neuronal cell loss or for the treatment of nerve cell or axonal degradation.

In a further aspect, the invention provides a method for the prevention of neuronal cell loss or for the treatment of nerve cell or axonal degradation, comprising administering to a patient an effective amount of the compound of Formula I as described herein.

In another aspect, the invention provides a commercial package containing the compound of Formula I as described herein, together with instruction for its use for the prevention of neuronal cell loss or for the treatment of nerve cell or axonal degradation.

### BRIEF DESCRIPTION OF THE DRAWINGS

Further aspects and advantages of the present invention will become better understood with reference to the description in association with the following Figures, wherein:

FIG. 1 is a graph showing the IC₅₀ inhibition curves of compound 23 with SHP1, PTP1B, LAR, cd45, PP1 and cathsepsin B. SHP1/2 phosphatase selectivity of compound 23 is demonstrated, with compound 23 displaying a distinct selectivity for SHP1 with an IC₅₀ of 10.6 μM.

FIG. 2 is an autoradiograph demonstrating TrkA phosphorylation with compound 23: The addition of NGF induces TrkA phosphorylation. The addition of compound 23 maintains the level of TrkA phosphorylation even after 24 hours. Lane PC12 624 cells not treated with NGF; Lane B—PC12 624 cells treated with NGF (50 ng/mL) for 24 hrs; Lane C—PC12 624 cells treated with NGF (50 ng/mL) for 30 minutes; Lane D—PC12 624 cells treated with NGF (10 ng/mL) and compound 23 (50 μM); Lane E—PC12 624 cells treated with NGF (10 ng/mL) and compound 23 (40 μM), Lane F—PC12 624 cells treated with NGF (10 ng/mL) and compound 23 (30 μM); Lane G—PC12 624 cells treated with NGF (10 ng/mL) and compound 23 (20 μM); Lane H—PC12 624 cells treated with a NGF (10 ng/mL) and compound 23 (10 μM); Lane I—PC12 624 cells treated with NGF (50 ng/mL) for 24 hours, and re-treated with NGF (50 ng/mL) for 30 minutes; Lane J—PC12 624 cells treated with NGF (50 ng/mL) for 30 minutes.

FIG. 3 shows Neurite Out-Growth induced in SCGs by Compound 23. FIG. 3A is an SCG ex-plant cultured in the absence of NGF. No neurite extensions are visible. FIG. 3B. is an SCG ex-plant cultured in the presence of compound 23 (30 μM). Numerous ordered neurites are present, extending 2-3 fields past that shown.

### DETAILED DESCRIPTION OF THE INVENTION

The compounds represented by Formula (I) may also be referred to as Compound (I) herein.

Unless otherwise stated, the following definitions apply either alone or in combination with another radical, C(1-8) alkyl means a straight-chain or branched alkyl group having 1 to 8 carbon atoms, such as methyl, ethyl, propyl, iso-propyl, n-butyl, iso-butyl, sec-butyl, tert-butyl, pentyl, iso-amyl, neopentyl, 1-ethylpropyl, hexyl, and octyl. The C(1-8)alkyl moiety of C(1-8)alkoxy, C(1-8)alkylsulfonyl, C(1-8)alkoxylcarbonyl, C(1-8)alkylaminocarbonyl has the same meaning as C(1-8)alkyl defined above. The acyl moiety of the acyl and the acyloxy group means a straight-chain or branched alkanoyl group having 1 to 18 carbon atoms, such as acetyl, propanoyl, butyryl, valeryl, pivaloyl and hexanoyl, and arylcarbonyl group described below, or a heteroarylcarbonyl group described below. The aryl moiety of the aryl, the arylcarbonyl and arylaminocarbonyl groups means a group having 6 to 16 carbon atoms such as, but not limited to, phenyl, biphenyl, naphthyl, or pyrenyl. The heteroaryl moiety of the heteroaryl and the heteroarylcarbonyl groups contain at least one hetero atom from O, N, and S, such as, but not limited to pyridyl, pyrimidyl, pyrroleyl, furyl, benzofuryl, thienyl, benzothienyl, imidazolyl, triazolyl, quinolyl, iso-quinolyl, benzoimidazolyl, thiazolyl, benzothiazolyl, oxazolyl, and indolyl. The aralkyl moiety of the aralkyl and the aralkyloxy groups having 7 to 15 carbon atoms, such as, but not limited to, benzyl, phenethyl, benzhydryl, and naphthylmethyl. The heteroaralkyl moiety of the heteroaralkyl and the heteroaralkyloxy groups having 7 to 15 carbon such as, but not limited to, pyridylmethyl, quinolinylmethyl, and iso-quinolinylmethyl. The heterocyclic group formed with a nitrogen atom includes rings such as, but not limited to, pyrrolyl, piperidinyl, piperidino, morpholinyl, morpholino, thiomorpholino, N-methylpiperazinyl, indolyl, and isoindolyl. The cycloalkyl moeity means a cycloalkyl group of the indicated number of carbon atoms, containing one or more rings anywhere in the structure, such as cycloalkyl groups include cyclopropyl, cyclopropylmethyl, cyclobutyl, cyclopentyl, cyclohexyl, 2-norbornyl, 1-adamantyl and the like. The fluoroalkyl moiety means a lower fluoroalkyl group in which one or more hydrogens of the corresponding C(1-8)alkyl group, as defined above, is replaced by a fluorine atom, such as but not limited to CH₂F, CHF₂, CF₃, CH₂CF₃, and CH₂CH₂CF₃.

Substituents may be, but are not limited to, C(1-8)alkyl, hydroxyl, C(1-8)alkoxy, C(1-8)alkylamino, dioxolane, dioxane, dithiolane, dithione, carboxyl, C(1-8)alkoxycarbonyl, nitro, amino, mono or di-C(1-8)alkylamino, azido, and halogen.

The substituted C(1-8)alkyl group can have 1 to 3 independently-selected substituents. The substituted aryl, the substituted heteroaryl, the substituted aralkyl, and the substituted heteroaralkyl groups each can have 1 to 5 independently-selected substituents.

Some of the compounds described herein contain one or more chiral centres and may thus give rise to diastereomers and optical isomers. The present invention is meant to comprehend such possible diastereomers as well as their racemic, resolved and enantiomerically pure forms, and pharmaceutically acceptable salts thereof.

The term “subject” or “patient” as used herein may refer to mammals including humans, primates, horses, cows, pigs, sheep, goats, dogs, cats, rodents, and the like.

The pharmaceutical compositions of the invention are administered to subjects in effective amounts. An effective amount means that amount necessary to delay the onset of, inhibit the progression of, or halt altogether the onset or progression of, or diagnose the particular condition or symptoms of, the particular condition being treated.

An effective amount for treating a neurological disorder is that amount necessary to affect any symptom or indicator of the condition, and/or reverse, halt or stabilize neuronal degradation and/or cell loss that is responsible for the particular condition being treated. In general, an effective amount for treating neuropathies and neuropathic pain will be that amount necessary to favorably affect the neuropathies and/or neuropathic pain. For example, an effective amount for treating neurodegenerative disease of the CNS, such as Alzheimer's disease is an effective amount to prevent memory loss, but is not limited to the amelioration of any one symptom. Similarly, an effective amount for treating Parkinson's disease or amyotrophic lateral sclerosis (ALS) is an amount necessary to favorably effect loss of muscular function and/or control, but is not limited to the amelioration of any one symptom. An effective amount for treating glaucoma and macular degeneration is an effective amount to prevent loss of vision. An effective amount for treating a peripheral neuropathy is an effective amount for preventing the development or halting the progression of PNS sensory or motor nerve dysfunction, but is not limited to these symptoms or effects.

When administered to a subject, effective amounts will depend, of course, on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation. It is preferred generally that a maximum dose be used, that is, the highest safe dose according to sound medical judgment.

A variety of administration routes are available. The particular mode selected will depend, of course, upon the particular condition being treated, the particular drug selected, the severity of the condition being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal, intradermal or parenteral routes. The term “parenteral” includes subcutaneous, intravenous, intramuscular, or infusion. Oral routes are preferred.

Dosage may be adjusted appropriately to achieve desired drug levels, locally or systemically. Generally, daily oral doses of active compounds will be from about 0.01 mg/kg per day to 1000 mg/kg per day. It is expected that IV doses in the range of about 1 to 1000 mg/kg per day will be effective. In the event that the response in a subject is insufficient at such doses, even higher doses (or effective higher doses by a different, more localized delivery route) may be employed to the extent that patient tolerance permits.

The compositions may conveniently be presented in unit dosage form and may be prepared by any of the methods well known in the art of pharmacy. All methods include the step of bringing the conjugates of the invention into association with a carrier that constitutes one or more accessory ingredients. In general, the compositions are prepared by uniformly and intimately bringing the compounds into association with a liquid carrier, a finely divided solid carrier, or both, and then, if necessary, shaping the product.

Compositions suitable for oral administration may be presented as discrete units such as capsules, cachets, tablets, or lozenges, each containing a predetermined amount of the active compound. Other compositions include suspensions in aqueous liquors or non-aqueous liquids such as a syrup, an elixir, or an emulsion.

Other delivery systems can include time-release, delayed release or sustained release delivery systems. Such systems can avoid repeated administrations of the active compounds of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. In addition, a pump-based hardware delivery system can be used, some of which are adapted for implantation.

A long-term sustained release implant also may be used. “Long-term” release, as used herein, means that the implant is constructed and arranged to deliver therapeutic levels of the active ingredient f6r at least 30 days, and preferably 60 days. Long-term sustained release implants are well known to those of ordinary skill in the art and include some of the release systems described above. Such implants can be particularly useful in treating solid tumors by placing the implant near or directly within the tumor, thereby affecting localized, high-doses of the compounds of the invention.

When administered, the Formulations of the invention are applied in pharmaceutically acceptable compositions. Such preparations may routinely contain salts, buffering agents, preservatives, compatible carriers, and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmaceutically acceptable salts include, but are not limited to, those prepared from the following acids: hydrochloric, hydrobromic, sulphuric, nitric, phosphoric, maleic, acetic, salicylic, p-toluenesulfonic, tartaric, citric, methane sulfonic, formic, malonic, succinic, naphthalene-2-sulfonic, benzene sulfonic, and the like. Also, pharmaceutically acceptable salts can be prepared as alkaline metal or alkaline earth salts, such as sodium, potassium or calcium salts.

Suitable buffering agents include: phosphate buffers, acetic acid and a salt (1-2% W/V); citric acid and a salt (1-3% W/V); and phosphoric acid and a salt (0.8-2% W/V), as well as others known in the art.

Suitable preservatives include benzalkonium chloride (0.003-0.03% W/V); chlorobutanol (0.3-0.9% W/V); parabens (0.01-0.25% W/V) and thimerosal (0.004-0.02% W/V), as well as others known in the art.

Suitable carriers are pharmaceutically acceptable carriers. The term pharmaceutically acceptable carrier means one or more compatible solid or liquid filler, dilutants or encapsulating substances that are suitable for administration to a human or other animal. The term “carrier” denotes an organic or inorganic ingredient, natural or synthetic, with which the active ingredient is combined to facilitate the application. The components of the pharmaceutical compositions are capable of being commingled with the molecules of the present invention, and with each other, in a manner such that there is no interaction which would substantially impair the desired pharmaceutical efficacy. Carrier formulations suitable for oral, subcutaneous, intravenous, and intramuscular administration etc., are those which are known in the art.

The compounds of the invention may be delivered with other therapeutic agents. The invention additionally includes co-administration of compound I of the invention with other compounds known to be useful in treating neurodegenerative diseases, typified by but not limited to, acetylcholinesterase inhibitors for treating AD, such as tacrine, doneprizil, and rivastigmin, and L-dopa for treating PD, and angiotensin-converting enzyme inhibitors (ACE inhibitors) and insulin for the treatment of diabetes.

In the case of peripheral neuropathy induced by a toxic agent, compound I would be delivered separately before, simultaneously with (ie. in the form of anti-cancer cocktails, see below), or after exposure to the toxic agent. Preferably, compound I and the chemotherapeutic agent are each administered at effective time intervals, during an overlapping period of treatment in order to prevent or restore at least a portion of the neurologicalfiinction destroyed by the neurotoxic or chemotherapeutic agent. The chemotherapeutic can be any chemotherapeutic agent that causes neurotoxicity, such as dideoxyinosine, deoxy cytizine, D4T, cisplatin, etoposide, vincristine, epithilone or its derivatives, or Taxol™/Taxoter™ and derivatives thereof, which are representative of the classes of agents which induce neuropathies.

By “toxic agent” or “neurotoxic agent” is meant a substance that through its chemical action injures, impairs, or inhibits the activity of a component of the nervous system. The list of neurotoxic agents that cause neuropathies is lengthy (see a list of candidate agents provided in Table 1). Such neurotoxic agents include, but are not limited to, neoplastic agents such as vincristine, vinblastine, cisplatin, Taxol™, or dideoxy—compounds, eg., dideoxyinosine; alcohol; metals; industrial toxins involved in occupational or environmental exposure; contaminants in food or medicinals; or over-doses of vitamins or therapeutic drugs, eg. Antibiotics such as penicillin or chloramphenicol, or mega-doses of vitamins A, D, or B6.

TABLE 1
Neurotoxic Agents

| AGENT | ACTIVITY |
|---|---|
| acetazolimide | diuretic |
| acrylamide | flocculant, grouting |
| | agent |
| adriamycin | antineoplastic |
| alcohol (ie. ethanol) | solvent, recreational drug |
| almitine | respiratory stimulant |
| amiodarone | antiarrthymic |
| amphotericin | antimicrobial |
| arsenic | herbicide, insecticide |
| aurothioglucose | antirheumatic |
| barbiturates | anticonvulsive, sedative |
| buckthorn | toxic berry |
| carbamates | insecticide |
| carbon disulfide | industrial applications |
| chloramphenicol | antibacterial |
| chloroquine | antimalarial |
| chlorestyramine | antihyperlipoproteinemic |
| cisplatin | antineoplastic |
| clioquinol | amebicide, antibacterial |
| colestipol | antihyperlipoproteinemic |
| colchicine | gout suppressant |
| colistin | antimicrobial |
| cycloserine | antibacterial |
| cytarabine | antineoplastic |
| dapsone | dermatological including |
| | leprosy |
| dideoxycytidine | anatineoplastic |
| dideoxyinosine | antineoplastic |
| dideoxythymidine | antiviral |
| disulfiram | antialcohol |
| doxorubicin | antineoplastic |
| ethambutol | antibacterial |
| ethionamide | antibacterial |
| glutethimide | sedative, hypnotic |
| gold | antirheumatic |
| hexacarbons | solvents |
| hormonal | |
| contraceptives | |
| hexamethylolmelamine | fireproofing, crease |
| | proofing |
| hydralazine | antihypertensive |
| hydroxychloroquine | antirheumatic |
| imipramine | antidepressant |
| indomethacin | anti-inflammatory |
| inorganic lead | toxic metal in |
| | paint, etc. |
| iso-niazid | antituberculousis |
| Lithium | antidepressant |
| methylmercury | industrial waste |
| metformin | antidiabetic |
| methylhydrazine | synthetic |
| | intermediate |
| metronidazole | antiprotozoal |
| misonidazole | radiosensitizer |
| nitrofurantoin | urinary antiseptic |
| nitrogen mustard | antineoplastic, |
| | nerve gas |
| nitous oxide | anesthetic |
| organophosphates | insecticides |
| Ospolot | anticonvulsant |
| penicillin | antibacterial |
| perhexiline | antiarrhythmic |
| perhexiline maleate | antiarrythmic |
| phenytoin | anticonvulsant |
| Platnim | drug component |
| primidone | anticonvulsant |
| procarbazine | antineoplastic |
| pyridoxine | vitamin B6 |
| Sodium cyanate | antisickling |
| streptomycin | antimicrobial |
| sulphonamides | antimicrobial |
| Suramin | anteneoplastic |
| tamoxifen | antineoplastic |
| Taxol ™ | antineoplastic |
| thalidomide | antileprous |
| Thallium | rat poison |
| triamterene | diuretic |
| trimethyltin | toxic metal |
| L-trypophan | health food |
| | additive |
| vincristine | antineoplastic |
| vinblastine | antineoplastic |
| vindesine | antineoplastic |
| vitamin A or D | mega doses |

Several neurotoxic agents and protocols may be used to induce apoptosis in SCG neurons. Several of these insults include the withdrawal of trophic support (for example NGF), treatment with neurotoxic chemotherapeutics such as Taxol™, cisplatin, vincristine, or vinblastine, and treatment with neurotoxic anti-virals. Selected compounds represented by Formula I have been found to inhibit apoptosis induced by the above insults.

Select compounds represented by Formula I protect cultured SCG neurons from several neurotoxic insults, including NGF withdrawal, treatment with anti-NGF anti-body, and treatment with chemotherapeutics such as Taxol™, cisplatin, and vincristine.

Neurotrophins are critical to the growth, development, and survival of small fiber neurons of the PNS. SCG neurons are neurons of the PNS that undergo apoptosis upon NGF withdrawal. In a typical experiment SCG neurons are cultured in the presence of NGF, which induces survival and neurite out-growth. After 5 days the NGF is removed by either the addition of anti-NGF polyclonal antibody (Sigma) or by repeated washings (4 times) with NGF free media, resulting in the apoptosis of up to 90% of the neurons after 48 hours, as measured by MTS staining. The addition of selected compounds of Formula I to the final cellular media provides upwards of 100% protection, at drug concentrations ranging from 10 to 50 μM (see Example 62).

Taxol™ is regularly used in the treatment of breast and other cancers. Taxol™ binds to the cyto-skeletal protein tubulin, thereby inhibiting normal microtubular assembly and inducing cellular apoptosis. Despite its potency as an anti-tumour agent, Taxol™ is also toxic to neurons, inducing dose limiting peripheral neuropathies. The addition of Taxol™ to cultured SCG neurons induces the degradation or loss of upwards of 80% of the neurons. The addition of selected compounds of Formula I to the cellular media, concurrently with Taxol™, protects upwards of 100% of the neurons, at drug concentrations ranging from 3 to 50 μM (see Example 63).

The mechanism of cisplatin's anti-cancer action is not fully understood, but is believed to involve DNA binding and cleavage. Cisplatin is toxic to neurons. The addition of cisplatin (3 μg/mL) to cultured SCG neurons induces apoptosis of upwards of 80% of the neurons. The addition of selected compounds of Formula I to the cellular media, concurrently with cisplatin, protects upwards of 100% of the neurons, at drug concentrations ranging from 1 to 50 μM (see Example 64).

NGF plays a critical and dose dependent role in the health of neurons of the PNS. For example, cultured SCG and DRG neurons will not survive in the absence of NGF. When the culture medium is supplemented with ‘minimal’ amount of NGF (1 ng/mL) the cell bodies remain viable, but limited or no neurite outgrowth is observed. When cultured in ‘optimal’ levels of NGF (50 ng/mL) the neurons remain healthy and produce robust neurite organizations. Similarly, the degree of TrkA phosphorylation observed in various neuronal cell types (SCGs, DRGs, PC12 cells which express TrkA) correlates well to the concentration of NGF (basal induction at 1 ng/mL, maximal induction at 50 ng/mL). Adeno-virus over expression of SHP1 reduces TrkA phosphorylation while dnSHP1 enhances TrkA phosphorylation. Therefore, it has been concluded that NGF induces cell survival and neurite extension by binding to and activating TrkA (inducing auto-phosphorylation). SHP1 dephosphorylates TrkA, inhibiting both its pro-growth and pro-survival functions. SHP2 performs a similar role in cortical neurons, binding to and dephosphorylating Trk B.

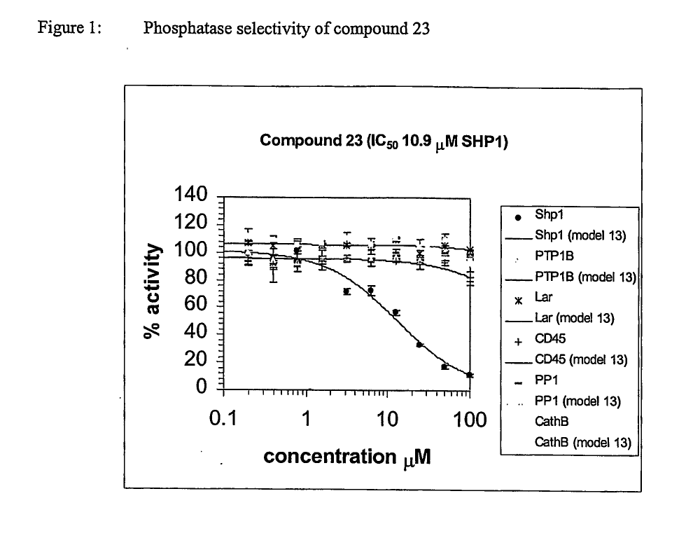

Select compounds represented by Formula I have displayed the ability to inhibit SHP1 and SHP2 phosphatases. In order to determine if these compounds represent general or selective phosphatase inhibitors they were tested against a panel of biologically important phosphatases, specifically PTP-1B, LAR, CD45, and PP1. Select compounds represented by Formula I inhibit SHP1 and SHP2 at concentrations ranging from 5 to 40 μM, with selectivity for SHP1, while displaying no or limited inhibition of PTP-1B, LAR, CD45, and PP1 (see example 65 and FIG. 1). These compounds represent selective SHP1/2 phosphatase inhibitors.

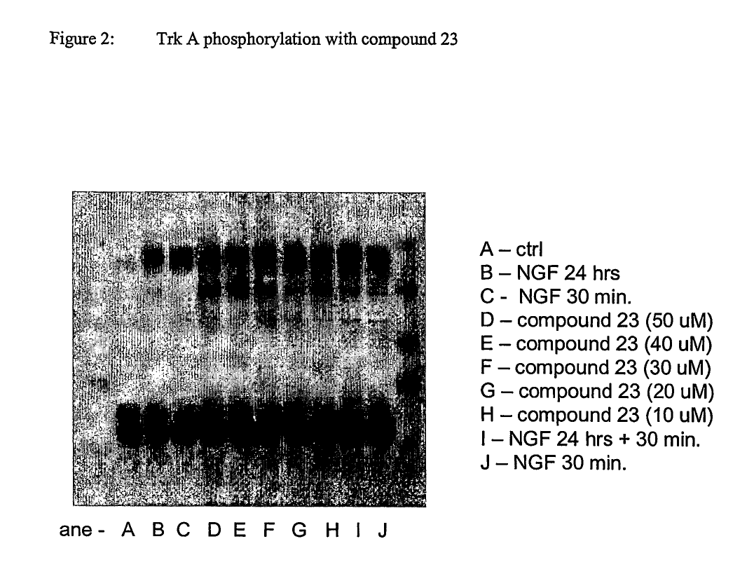

TrkA phosphorylation may be monitored using Trk anti-bodies and Western blot analysis. In this way TrkA phosphorylation is dramatically increased by the presence of NGF in both a time and concentration dependent manner. In PC12 expressing cells, this effect is most robust at 30 minutes when 50 ng/mL of NGF is used. Comparable levels of TrkA phosphorylation was observed when PC12 cells were treated with 10-50 μM of compound 23 (see Example 66 and FIG. 2).

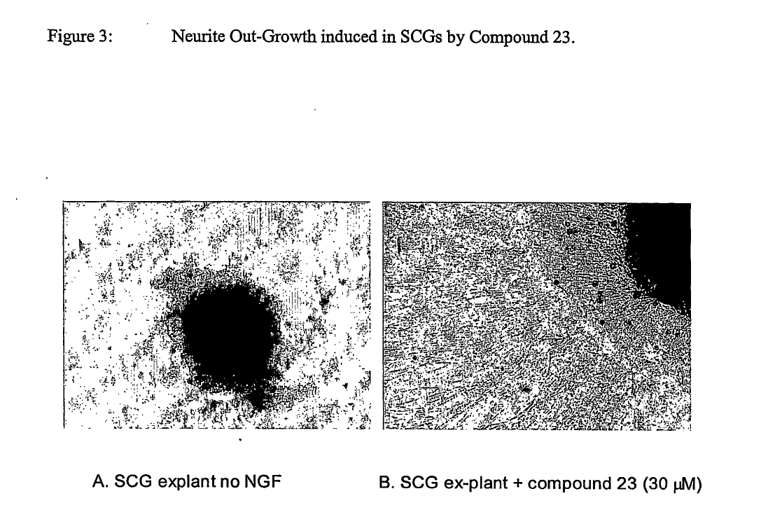

In order to determine if these compounds were capable of augmenting NGF signaling in vitro, compound was added to cultured SCG neurons in the presence of ‘minimal’ quantities of NGF. As mentioned above, SCG ex-plants cultured in media containing 1 ng/mL NGF remain viable but do not produce neurites. In comparison, when media containing a 1 ng/mL NGF and 30 μM of compound 23 was applied to freshly cultured SCG neurons neurite outgrowth, after 2 days, was visibly comparable to that observed under ‘optimal’ NGF concentrations (50 ng/mL) (see FIG. 3). Similar results were obtained using NGF differentiated PC12 cells (not shown).

The phenolic moiety observed in many of the compounds represented by Formula I may be converted to esters, carbamates, thiocarbamates and sulfonates. The Applicant has shown that both esters and carbamates are rapidly converted to the corresponding phenol when incubated in the presence of microsomal fractions using standard conditions (Cresteil, T., et al. Am. Soc. Pharm. Exper. Therapeutics, 2002, 30, 438-445). These microsomal fractions contain various active esterase and P450 oxidative enzymes. In this way, compounds 25 and 55 are rapidly converted to their parent phenols, compounds 23 and 54, respectively. Esters and carbamates of this type allow for the preparation of various novel pro-drugs which display increased water solubility, stability, formulation, and pharmacokinetic profiles. The carbamates disclosed in U.S. Pat. No. 4,349,685 are limited to lower alkyl, substituted aryl, haloalkyl, carbalkoxyalkene or an alkyl carbonyloxyalkylene group. The preferred embodiments of this disclosure include esters and carbamates represented by Formula I, where the carbamate or ester moiety includes substituted lower alkyl, heteroaryl, substituted heteroaryl moieties which contain a basic nitrogen, capable of increasing the water solubility of said compound via an acid/base salt (typified by compounds 55 and 61).

Taken together, select compounds represented by Formula I display significant neuroprotective profiles. This extends both to neurons of the PNS and the CNS. Additionally, select compounds represented by Formula I represent inhibitors of SHP1/2 phosphatases which have been shown to positively affect NGF signaling in neurons. Carbamate and acetoxy derivatives of this class of molecules are rapidly metabolized allowing for the design of novel pro-drugs of select compounds represented by Formula I, which allows for the tuning of solubility and pharmacokinetic profiles.

Compounds represented by formula I are included in Table 2.

TABLE 2
Compounds represented by formula I

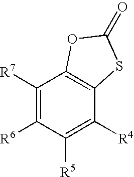

| compound | R⁴ | R⁵ | R⁶ | R⁷ |
|---|---|---|---|---|
| 1 | H | OH | H | H |
| 2 | H | 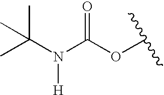 | H | H |
| 3 | Cl | OH | H | Cl |
| 4 | Cl | 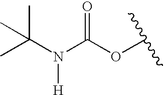 | H | Cl |
| 5 | H | OH | H | —SCH₂CH₃ |
| 6 | H | OH | H | —S(O)CH₂Ph |
| 7 | H | OH | H | —SCH₂CH₂Ph |
| 8 | H | OH | H | —SPh |
| 9 | H | OH | H | 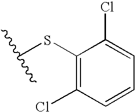 |
| 10 | H | OH | 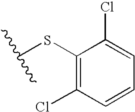 | H |
| 11 | H | OH | H | 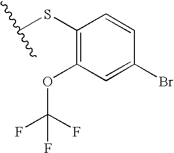 |
| 12 | H | OH | H | 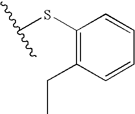 |
| 13 | H | OH | H | 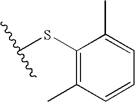 |
| 14 | H | OH | H | 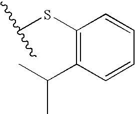 |
| 15 | H | OH | 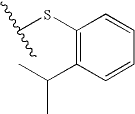 | H |
| 16 | H | OH | H | 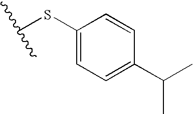 |
| 17 | H | OH | H | 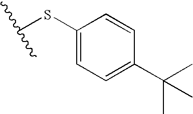 |
| 18 | H | OH | H | 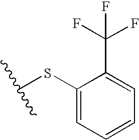 |
| 19 | H | OH | H | 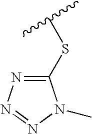 |
| 20 | 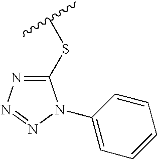 | OH | H | H |
| 21 | 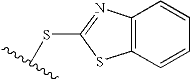 | OH | H | H |
| 22 | H | OH | H | 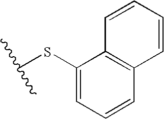 |
| 23 | H | OH | H | 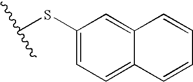 |
| 24 | H | OH | H | 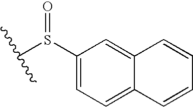 |
| 25 | H | 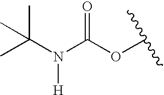 | H | 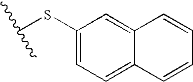 |
| 26 | H | 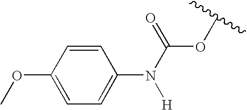 | H | 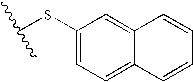 |
| 29 | 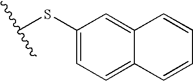 | OH | H | PhS— |
| 30 | 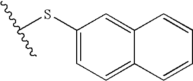 | OH | H | 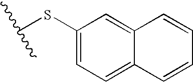 |
| 31 | H | 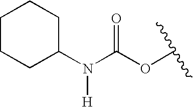 | H | —CH₃ |
| 32 | H | OH | H | —Ph |
| 33 | H | 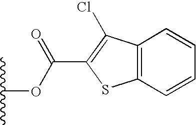 | H | —Ph |
| 34 | H | 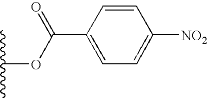 | H | —Ph |
| 35 | H | OH | H | 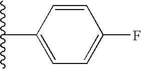 |
| 36 | H | OH | H | 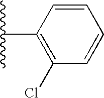 |
| 37 | H | OH | H | 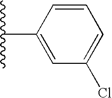 |
| 38 | H | 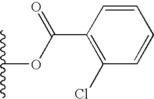 | H | 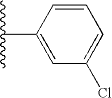 |
| 39 | H | OH | H | 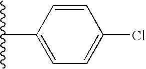 |
| 40 | H | 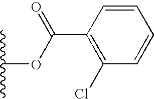 | H | 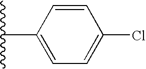 |
| 41 | H | OH | H | 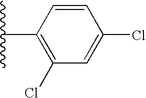 |
| 42 | H | OH | H | 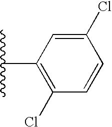 |
| 43 | H | OH | H | 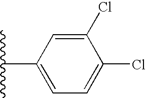 |
| 44 | H | OH | H | 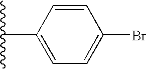 |
| 45 | H | OH | H | 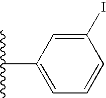 |
| 46 | H | OH | H | 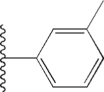 |
| 47 | H | OH | H | 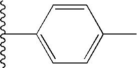 |
| 48 | H | 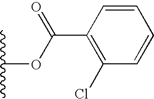 | H | 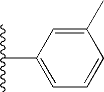 |
| 49 | H | OH | H | 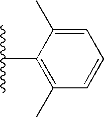 |
| 50 | H | OH | 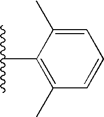 | H |
| 51 | H | OH | H | 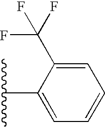 |
| 53 | H | OH | H | 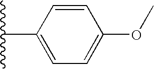 |
| 54 | H | OH | H | 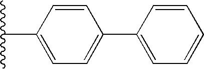 |
| 55 | H | 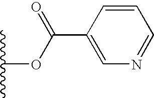 | H | 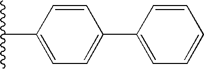 |
| 56 | H | CH₃— | H | CH₃CH₂NH— |
| 57 | H | R5, R6, and R7 together form —(CH₂)₃N(CH₂)₃— | | |
| 58 | H | 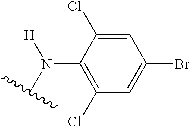 | H | H |
| 59 | H | 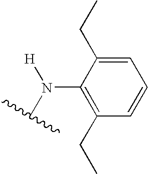 | H | H |
| 60 | napthyltbio | OH | R6 and R7 together form a fused | |
| | | | phenyl ring | |
| 61 | H | 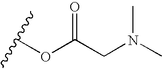 | H | 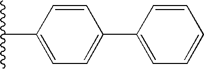 |

Synthetic Methods

The desired benzo[1,3]oxathiol-2-ones were prepared from the corresponding benzoquinones by treatment with thiourea followed by NaOAc, using modifications to the procedure described by Lau, P. T. S. and Kestner, M.(Lau, P. T. S. and Kestner, M, J. Org. Chem., 1968, 33, 4426).

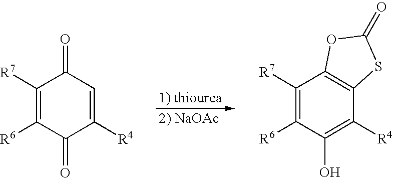

The requisite 2-alkylthio and 2-arylthio benzoquinones were prepared by treating 2 equivalents of benzoquinone with the appropriate thiol as described (Lau, P. T. S. and Kestner, M. J. Org. Chem., 1968, 33, 4426).

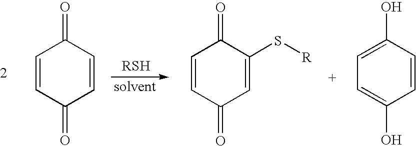

The requisite 2-arylbenzoquinones were prepared by treating benzoquinone with the appropriate aniline and NaNO₂ (Shimmelschmidt, K. Liebigs (1950), 566, 184; Bassard, P., L'Ecuyer, P., Can. J. Chem., 1958, 36, 709).

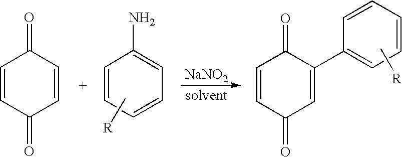

Treatment of 5-hyrdoxybenzo[1,3]oxathiol-2-ones with either an acyl halide, anhydride, or a DIC activated carboxylic acid provided the desired 5-(O-acetoxy)benzo[1,3]oxathiol-2-ones. Similarly, treatment with iso-cyanates provided the corresponding carbamates (U.S. Pat. No. 4,349,685).

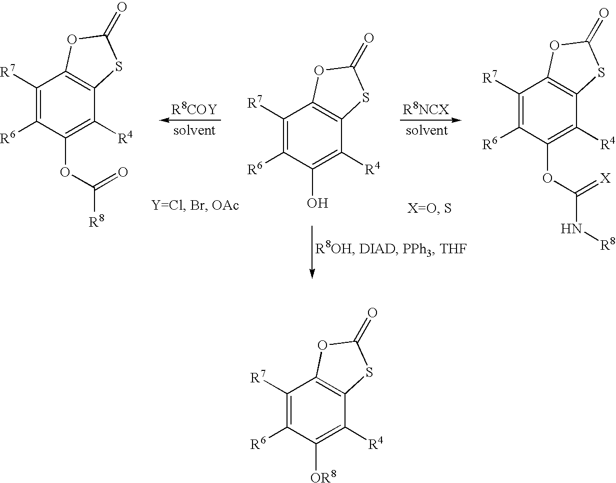

Direct alkylation of the 5-hydroxybenzo[1,3]oxathiol-2-ones was accomplished using Mitsunobu coupling with an appropriate alcohol (Elliot, R. L., et al. Bioorg. Med. Chem. Lett., 1996, 6, 2283).

### EXAMPLES

Abbreviations or symbols used herein include: CDCl₃—deuterated chloroform; DIC—diisopropylcarboxidimide; DIAD—diisopropyl azodicarboxylate; DMF—dimethylformamide; DMSO—dimethylsulfoxide; MgSO₄—magnesium sulphate; NMR—nuclear magnetic resonance; Ph₃P—triphenylphosphine; NaOAc—sodium acetate; NaNO₂—sodium nitrite; THF—tetrahydrofuran; tlc—thin layer chromatography.

The following non-limiting examples are illustrative of the present invention:

### Example 1

5-Hydroxybenzo[1,3]oxathiol-2-one. Compound 1 was prepared according to the procedure described by H. Burton and S. B. Davis, J. Org. Chem., 1952, 2193. ¹H NMR (200 MHz, CDCl₃) δ 9.75 (s, 1H), 7.24 (d, J=8.5 Hz, 1H), 7.10 (d, J=2.5 Hz, 1H), 6.74 (dd, J=8.5, 2.5 Hz, 1H). ¹³C NMR (50 MHz, CDCl₃) δ 169.5, 154.9, 140.8, 123.2, 114.6, 112.5, 109.5.

### Example 2

5-(N-Butylcarbamoyloxy)benzo[1,3]oxathiol-2-one. 5-Hydroxy-benzo[1,3]oxathiol-2-one (840 mg, 5 mmol) and butyl isocyanate (1.0 g, 10 mmol) were dissolved in DMF (5 mL). Triethylamine (500 mg, 5 mmol) was added and the mixture heated at 80° C. for 2 h. Solvent was removed under high vacuum and the residue purified by silica gel chromatography, eluting with 9:1 ethyl acetate/hexane, to provide a white solid (650 mg). ¹H NMR (200 MHz, CDCl₃) δ 7.23 (m, 2H), 7.05 (dd, J=2.5, 9.0 Hz, 1H), 3.25 (q, J=6.6 Hz, 2H), 1.55 (m, 2H), 1.40 (m, 2H), 0.94 (t, J=6.6 Hz, 3H). ¹³C NMR (50 MHz, CDCl₃) δ 167.9, 154.2, 148.1, 145.2, 123.6, 121.6, 116.1, 112.3, 41.0, 31.7, 19.7, 13.6.

### Example 3

4,7-Dichloro-5-hydroxybenzo[1,3]oxathiol-2-one. Thiourea (8.00 g, 105 mmol) was dissolved in a solution of water (32.0 mL), concentrated hydrochloric acid (8.0 mL) and ethanol (30.0 mL). To this vigorously stirred solution was then added solid 2,5-dichlorobenzoquinone (13.00 g, 73 mmol) in small portion over ca. 20 minutes allowing all the solid to dissolve before adding more. The reaction mixture was then stirred for an additional 30 minutes upon which concentrated hydrochloric acid (60 mL) was added. The resulting solid was allowed to settle for 12 hours and was filtered under vacuum and washed with small portion of concentrated hydrochloric acid. The wet solid was suspended in water (100 mL) to which was added acetic acid (40 mL) and concentrated hydrochloric acid (20 mL). The mixture was heated to reflux for 2 hrs. Volatiles were removed under reduced pressure and the residue was partitioned between ethyl acetate and water. The organic layer was dried over anhydrous MgSO₄, filtered and evaporated to dryness to provide crude compound 3. Purification by silica gel column chromatography (eluting with 10% ethyl acetate/hexane) provided compound 3 as a white solid (5.0 g, 30%). ¹H NMR (200 MHz, DMSO-d⁶) δ 11.00 (br s, 1H), 7.00 (s, 1H).

### Example 4

5-N-Butylcarbamoyloxy)-4,7-dichlorobenzo[1,3]oxathiol-2-one. 4,7-Dichloro-5-hydroxy-benzo[1,3]oxathiol-2-one (237 mg, 1 mmol) was dissolved in DMF (1 mL) to which was added butyl isocyanate (200 mg, 2 mmol) followed by triethylamine (100 mg, 1.0 mmol). The mixture was stirred at RT for 5 minutes before volatiles were removed under reduced pressure. Purification by silica gel chromatography (eluting with 9:1 ethyl acetate/hexane) provided compound 4 as a white fluffy solid (270 mg, 80%). ¹H NMR (200 MHz, DMSO-d⁶) δ 8.08 (t, J=9 Hz, 1H), 7.65 (s, 1H), 3.02 (q, J=8.0 Hz, 2H), 1.6-1.2 (m, 4H), 0.86 (t, J=8.0 Hz, 3H). ¹³C NMR (50 MHz, DMSO-d⁶) δ 165.5, 152.4, 144.1, 141.0, 124.5, 123.2, 118.7, 114.4, 39.9, 30.7, 18.9, 13.1.

### Example 5

7-Ethylsulfanyl-5-hydroxybenzo[1,3]oxathiol-2-one. Compound 5 was prepared as per compound 8. ¹H NMR (200 MHz, DMSO-d⁶) [symbol] 9.87 (bs, 1H), 6.97 (d, J=2.4 Hz, 1H), 6.72 (d, J=2.4 Hz, 1H), 2.97 (q, J=2.4 Hz, 2H), 1.23 (t, J=2.4 Hz, 3H).

### Example 6

Benzyl 5-hydroxy-7-(phenylmethanesulfinyl)benzo[1,3]oxathiol-2-one). Compound 6 was prepared as per compound 8. ¹H NMR (200 MHz, DMSO-d⁶) δ 10.12 (bs, 1H), 7.25 (m, 4H), 7.03 (m, 2H), 6.78(s, 1H), 4.252 (d, J=5.49 Hz, 2H).

### Example 7

5-hydroxy-7-(phenethylsulfanyl)benzo[1,3]oxathiol-2-one. Compound 7 was prepared as per compound 8. ¹H NMR (200 MHz, DMSO-d⁶) δ 9.98 (bs, 1H), 7.26 (m, 5H), 6.97 (s, 1H), 6.76(s, 1H), 3.24 (t, J=7.9 Hz, 2H), 2.87 (t, J=7.94 Hz, 2H).

### Example 8

5-hydroxy-7-(phenylsulfanyl)benzo[1,3]oxathiol-2-one. Step 1: 1-4-Benzoquinone (4.30 g, 40 mmol) was dissolved in ethanol (150 mL). A solution of thiophenol (2.05 mL, 20 mmol) in ethanol (5 mL) was added at once and the mixture vigorously stirred. Additional 1,4-benzoquinone (2.70 g, 25 mmol) was added. The solution was cooled on ice prior to filtration, washing with cold ethanol, to provide 2-phenylthio[1,4]benzoquinone as an orange solid (3.48 g, 80%). ¹H NMR (200 MHz, CDCl₃) δ 7.56-7.42 (m, 5H), 6.8 (d, J=10.0 Hz, 1H), 6.6 (dd, J=10, 3 Hz, 1H), 5.8 (d, J=3 Hz, 1H). Step 2: A solution of thiourea (1.10 g, 14 mmol) in 2N HCl (5 mL) was diluted with ethanol (5 mL). Solid 2-(phenylthio)[1,4]benzoquinone (1.80 g, 8.3 mmol) was added in portions while heating. Ethanol and 2N HCl were added such as to preserve suitable reaction fluidity (up to a total of 150 mL). Upon completion of the reaction (as determined by tlc), conc. HCl was added and the resulting solid filtered and then suspended in conc. HCl (20 mL) and acetic acid (40 mL). This mixture was heated at reflux for 2 h. The reaction mixture was evaporated to dryness and then chased with toluene. The resulting crude material was purified by silica gel chromatography (eluting with 20% ethyl acetate/hexane) to provide compound 8 as a white solid (900 mg). ¹H NMR (200 MHz, DMSO-d⁶) δ 9.95 (s, 1H), 7.4 (m, 5H), 7.05 (s, 1H), 6.45 (s, 1H). ¹³C NMR (50 MHz, CDCl₃) [symbol] 168.8, 155.2, 138.9, 131.85, 131.8, 129.9, 128.4, 123.9, 119.7, 115.8, 108.9.

### Examples 9 and 10

7-((2,6-Dichlorophenyl)sulfanyl)-5-hydroxybenzo[1,3]oxathiol-2-one and 6-((2,6-Dichlorophenyl)sulfanyl)-5-hydroxybenzo[1,3]oxathiol-2-one. Compounds 9 and 10 were prepared as per compound 8, yielding the two regeoisomers. These compounds were separated by silica gel chromatography. Compound 9: ¹H NMR (200 MHz, CDCl₃) δ 7.44 (d, 1H), 7.24 (m, 2H), 6.64 (d, J=1.5 Hz, 1H), 6.14 (d, J=1.5 Hz, 1H). LCMS (API-ES, neg. scan, m/z) M-1=344. Compound 10: LCMS (API-ES, neg. scan, m/z) M-1=344.

### Example 11

5-hydroxy-7-((4-Bromo-2-trifluoromethoxypheny)sulfanyl)benzo[1,3]oxathiol-2-one. Compound 11 was prepared as per compound 8. LCMS (API-ES, neg. scan, m/z) M-1=438.

### Example 12

7-((2-Ethylphenyl)sulfanyl)-5-hydroxybenzo[1,3]oxathiol-2-one. Compound 12 was prepared as per compound 8. ¹H NMR (200 MHz, CDCl₃) δ 7.46 (d, J=7.4 Hz, 1H), 7.37 (m, 2H), 7.20 (m, 1H), 6.66 (d, J=2.4 Hz, 1H), 6.11 (d, J=2.4 Hz, 1H), 4.80 (bs, 1H), 2.81 (q, J=7.6 Hz, 2H), 1.21 (t, J=7.6 Hz, 3H).

### Example 13

7-((2,6-Dimethylphenyl)sulfanyl)-5-hydroxybenzo[1,3]oxathiol-2-one. Compound 13 was prepared as per compound 8. LCMS (API-ES, neg. scan, m/z) M-1=303.

### Example 14

5-hydroxy-7-((2-iso-Propylphenyl)sulfanyl)benzo[1,3]oxathiol-2-one. Compound 14 was prepared as per compound 8. ¹H NMR (200 MHz, CDCl₃) δ 7.21 (m, 4H), 6.65 (d, J=2.3 Hz, 1H), 6.12 (d, J=2.4 Hz, 1H), 3.52 (hept, J=6.9 Hz, 1H), 1.20 (d, J=6.9 Hz, 6H).

### Example 15

5-hydroxy-6-((2-iso-Propylphenyl)sulfanyl)benzo[1,3]oxathiol-2-one. Compound 15 was prepared as per compound 8. ¹H NMR (200 MHz, CDCl₃) [symbol] 7.36 (s, 1H), 7.32 (dd, 1H), 7.20 (t, 1H), 7.12 (s 1H), 7.02 (t, 1H), 6.74 (d, 1H), 6.44 (br s, 1H), 3.52 hept, J=6.8 Hz, 1H), 1.20 (d, J=6.8 Hz, 6H).

### Example 16

7-(4-iso-Propylphenylthio)-5-hydroxybenzo[1,3]oxathiol-2-one. Compound 16 was prepared as per compound 8. ¹H NMR (200 MHz, CDCl₃) δ 9.87 (s, 1H), 7.34 (m, 4H), 7.02 (d, J=3 Hz, 1H), 6.36 (d, J=3 Hz, 1H), 2.90 (hept, J=6 Hz, 1H), 1.18 (d, J=6 Hz, 6H).

### Example 17

7-((4-tert-Butylphenyl)sulfanyl)-5-hydroxybenzo[1,3]oxathiol-2-one. Compound 17 was prepared as per compound 8. ¹H NMR (200 MHz, CDCl₃)δ 9.88 (s, 1H), 7.41 (m, 4H), 7.03 (d, J=6 Hz, 1H), 6.37 (d, J=6 Hz, 1), 1.26 (s, 9H).

### Example 18

5-hydroxy-7-((2-Trifluoromethylphenyl)sulfanyl)benzo[1,3]oxathiol-2-one. Compound 18 was prepared as per compound 8. ¹H NMR (200 MHz, CDCl₃) δ 7.8 (d, 1H), 7.65 (m, 2H), 7.40 (d, 1H), 7.01 (d, J=2.2 Hz, 1H), 6.66 (d, J=2.2 Hz, 1H).

### Example 19

5-Hydroxy-7-((N-methyltetrazol-2-yl)sulfanyl)benzo[1,3]oxathiol-2-one. Compound 19 was prepared as per compound 8. ¹H NMR (200 MHz, DMSO-d⁶.)δ 10.79 (bs, 1H), 7.47 (d, J=8.8 Hz, 1H), 6.94 (d, J=8.8 Hz, 1H), 4.04 (s, 3H).

### Example 20

5-hydroxy-4-((N-phenyltetrazol-2-yl)sulfanyl)benzo[1,3]oxathiol-2-one. Compound 20 was prepared as per compound 8. ¹H NMR (200 MHz, DMSO-d) δ 7.64 (s, 5H), 7.44 (d, J=9.0 Hz, 1H), 6.90 (d, J=9.0 Hz, 1H).

### Example 21

4-((Benzothiazol-2-yl)sulfanyl)-5-hydroxybenzo[1,3]oxathiol-2-one. Compound 21 was prepared as per compound 8. ¹H NMR (200 MHz, DMSO-d) δ 7.92 (d, J=7.5 Hz, 1H), 7.84 (d, J=7.5 Hz, 1H), 7.61 (d, J=9.0 Hz, 1H), 7.45 (t, J=7.5 Hz, 1H), 7.33 (t, J=7.5 Hz, 1H), 7.11 (d, J=9.0 Hz, 1H).

### Example 22

5-Hydroxy-7-((1-naphthyl)sulfanyl)benzo[1,3]oxathiol-2-one. Compound 22 was prepared as per compound 8. ¹H NMR (200 MHz, CDCl₃)δ 9.71 (s, 1H), 8.08 (m, 3H), 7.81 (dd, J=2.0, 8.0 Hz, 1H), 7.62 (m, 3H), 6.95 (d, J=4.0 Hz, 1H), 6.01 (d, J=4.0 Hz, 1H).

### Example 23

5-Hydroxy-7-((2-naphthy)sulfanyl)benzo[1,3]oxathiol-2-one. Compound 23 was prepared as per compound 8. ¹H NMR (200 MHz, DMSO-d⁶) δ 9.88 (bs, 1H), 8.06 (s, 1H), 7.95 (m, 3H), 7.57 (m, 2H), 7.47 (d, 1H), 7.07 (d, J=2.4 Hz, 1H), 6.45 (d, J=2.4 Hz, 1H).

### Example 24

5-Hydroxy-7-((2-naphthyl)sulfinyl)benzo[1,3]oxathiol-2-one. Compound 23 was refluxed in MeOH open to air and allowed to crystallize to yield compound 24. ¹H NMR (200 MHz, DMSO-d⁶) δ 8.43 (bs, 1H), 8.05 (m, 3H), 7.64 (m, 3H), 7.28 (d, J=2.4 Hz, 1H), 7.14 (d, J=2.4 Hz, 1H).

### Example 25

5-(N-Butylcarbamoyloxy)-7-((2-naphthyl)sulfanyl)benzo[1,3]oxathiol-2-one. Compound 23 (100 mg, 0.31 mmol), n-butyl isocyanate (38 μL, 0.34 mmol) and triethylamine (12.8 μL, 0.10 mmol) in acetonitrile (10 mL) were heated at 70° C. for 2 hours and the solvents removed in vacuo. Standard ethyl acetate/water workup and purification by silica gel chromatography (eluting 5:1 ethyl acetate/hexane) provided compound 25 (45 mg, 42%) as an off white powder. ¹H NMR (DMSO-d⁶) δ 8.07 (s, 1H), 7.99-7.90 (m, 3H), 7.75 (t, J=5.5 Hz, 1H), 7.58-7.54 (m, 3H), 7.45 (dd, J=8.8, 1.4 Hz, 1H), 6.78 (dd, J=2.6, 0.6 Hz, 1H), 2.95 (q, J=6.8 Hz, 2H), 1.35-1.16 (m, 4H), 0.80 (t, J=6.8 Hz, 3H). ¹³C NMR (DMSO-d⁶) δ 168.6, 153.8, 148.2, 143.0, 133.5, 132.4, 131.2, 129.6, 128.7, 127.8, 127.7, 127.2 (2×), 124.0, 122.3, 119.8, 116.3, 40.1, 31.1, 19.3, 13.5.

### Example 26

5-(N-(4-methoxyphenyl)carbamoyloxy)-7-((2-naphthyl)sulfanyl)benzo[1,3]oxathiol-2-one. Compound 26 was prepared as per compound 25 using compound 23 and 4-methoxyphenyl isocyanate. LCMS (API-ES, neg. scan, m/z) M-1=474.

### Example 29

5-hydroxy-4-(2-naphthylthio)-7-((phenyl)sulfanyl)benzo[1,3]oxathiol-2-one: LCMS (API-ES, neg. scan, m/z) M-1=433.

### Example 30

4,7-Bis-((2-naphthyl)sulfanyl)-5-hydroxybenzo[1,3]oxathiol-2-one.

LCMS (API-ES, neg. scan, m/z) M-1=483.

### Example 31

5-(N-Cyclohexylcarbamoyloxy)-7-methylbenzo[1,3]oxathiol-2-one

### Example 32

4-Hydroxy-7-phenylbenzo[1,3]oxathiol-2-one. Compound 32 was prepared according to literature procedures (U.S. Pat. No. 2,332,418) using 2-phenylbenzoquinone and thiourea. LCMS (API-ES, neg. scan, m/z) M-1=243.

### Example 33

5-(3-chlorobenzothiophen-2-ylcarbonyloxy)-7-phenylbenzo[1,3]oxathiol-2-one

### Example 34

6-(4-nitrophenylcarbonyloxy)-7-phenylbenzo[1,3]oxathiol-2-one

### Example 35

7-(4-fluorophenyl)-5-hydroxybenzo[1,3]oxathiol-2-one

### Example 36

7-(2-chlorophenyl)-5-hydroxybenzo[1,3]oxathiol-2-one

### Example 37

7-(3-chlorophenyl)-5-hydroxybenzo[1,3]oxathiol-2-one

### Example 38

5-(2-chlorophenylcarbonyloxy)-7-(3-chlorophenyl)benzo[1,3]oxathiol-2-one

### Example 39

7-(4-chlorophenyl)-5-hydroxybenzo[1,3]oxathiol-2-one

### Example 40

5-(2-chlorophenylcarbonyloxy)-7-(4-chlorophenyl)benzo[1,3]oxathiol-2-one

### Example 41

7-(2,4-dichlorophenyl)-5-hydroxybenzo[1,3]oxathiol-2-one

### Example 42

7-(2,5-dichlorophenyl)-5-hydroxybenzo[1,3]oxathiol-2-one

### Example 43

7-(3,4-dichlorophenyl)-5-hydroxybenzo[1,3]oxathiol-2-one

### Example 44

7-(4-bromophenyl)-5-hydroxybenzo[1,3]oxathiol-2-one

### Example 45

5-hydroxy-7-(3-iodophenyl)benzo[1,3]oxathiol-2-one. Compound 45 was prepared as per compound 54. LCMS (API-ES, neg. scan, m/z) M-1=369.

### Example 46

5-hydroxy-7-(3-methylphenyl)benzo[1,3]oxathiol-2-one

### Example 47

5-hydroxy-7-(4-methylphenyl)benzo[1,3]oxathiol-2-one

### Example 48

5-(2-chlorophenylcarbonyloxy)-7-(3-methylphenyl)benzo[1,3]oxathiol-2-one

### Examples 49 and 50

7-(2,6-dimethylphenyl)-5-hydroxybenzo[1,3]oxathiol-2-one and 6-(2,6-dimethylphenyl)-5-hydroxybenzo[1,3]oxathiol-2-one. Compounds 49 and 50 were prepared as per compound 54 to provide the two regeoisomers which were separated by silica gel chromatography, eluting with 30-50% ethyl acetate/hexane. LCMS (API-ES, neg. scan, m/z) M-1=270.

### Example 51

5-hydroxy-7-(2-trifluoromethylphenyl)benzo[1,3]oxathiol-2-one. Compound 51 was prepared as per compound 54. LCMS (API-ES, neg. scan, m/z) M-1=270.

### Example 53

5-hydroxy-7-(4-methoxyphenyl)benzo[1,3]oxathiol-2-one

### Example 54

7-biphenyl-5-hydroxybenzo[1,3]oxathiol-2-one. 4-Aminobiphenyl (1.70 g, 10 mmol) in water (40 mL) was treated with conc HCl (2.5 mL). This solution was cooled on ice and sodium nitrate (0.71 g, 10 mmol) dissolved in a minimum amount of water was added drop wise, to provide the diazonium salt solution. The reaction was allowed to stir for approximately 5 minutes. 1,4-Benzoquinone (1.10 g, 10 mmol) was dissolved in ethanol and the diazonium salt solution was added. Sodium acetate (5 mg) was added until bubbles began to form. The reaction was allowed to stir for approximately 1 hour monitored by TLC (30% dichloromethane/toluene). A dark orange-brown precipitate formed which was filter filtered. The resulting brown solid was dried and chased with toluene to eliminate any remaining acetic acid to yield 2-biphenyl-1,4-benzoquinone (1.24 g, 47% yield). ¹H NMR (200 MHz, CDCl₃) [symbol] 7.56-7.71 (m, 5H), 7.38-7.51 (m, 4 H), 6.86-6.94 (m, 3H).

Thiourea (670 mg, 8.00 mmol) was dissolved in 2N HCl (30 mL). 2-Biphenyl-1,4-benzoquinone (1.15 g, 4.00 mmol) in ethanol (30 mL) was heated gently and added to a solution of thiourea (670 mg, 8.00 mmol) dissolved in 2N HCl (30 mL). The resulting brown suspension was allowed to stir for approximately 30 minutes. The mixture was then heated and allowed to evaporate to a thick brown paste. 20 mL of concentrated HCl and 20 mL of concentrated acetic acid was added to the brown paste and the reaction was refluxed for approximately 2 hours. The solution became a cloudy brown. The mixture was filtered and a light brown solid was collected, loaded on silica gel and purified by silica gel chromatography (eluting with 20% ethyl acetate/0.1% acetic acid/hexane) to provide compound 54 as pale pink solid (128 mg, 9%). ¹H NMR (200 MHz, DMSO-d⁶) δ 9.85 (s, 1H), 7.20-7.80 (m, 5H), 7.35-7.55 (m, 4H), 7.90 (d, 1 H), 6.90 (d, 1H).

### Example 55

5-(3-pyridylcarbonyloxy)-7-biphenylbenzo[1,3]oxathiol-2-one. Compound 54 (78 mg) was dissolved in THF (5 mL) and treated with triethylamine (67 mL) followed by nicotinoyl chloride hydrochloride (52 mg). The mixture was stirred overnight at room temperature under nitrogen. The mixture was then taken up in ethyl acetate (50 mL) and washed with 50 mL each of brine, NaHCO₃ (×2) and brine. The ethyl acetate layer was dried over anhydrous MgSO₄, filtered, and the solvent removed under reduced pressure to provide compound 55 (68 mg). ¹H NMR (200 MHz, CDCl₃)δ 8.46 (d, J=8.2 Hz, 1H), 7.73 (s, 4H), 7.64 (d, J=8.2 Hz, 2H), 7.4 (m, 7H).

### Example 56

7-ethylamino-5-methylbenzo[1,3]oxathiol-2-one

### Example 57

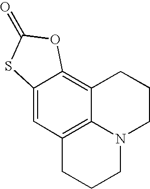

### Example 58

5-(4-bromo-2,6-dichloroanalino)benzo[1,3]oxathiol-2-one

### Example 59

6-(2,6-diethylanalino)benzo[1,3]oxathiol-2-one

### Example 60

4-Hydroxy-3-(2-naphthylthio)naphtha[2,1-d]1,3-oxathiol-2-one

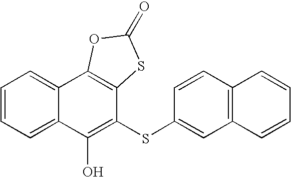

### Example 61

5-(N,N-dimethylaminomethylcarbonyloxy)-7-biphenylbenzo[1,3]oxathiol-2-one. As per compound 55. LCMS (API-ES, neg. scan, m/z) M-1=404.

### Example 62

Protection of SCG neurons from anti-NGF killing. SCG neurons were isolated from day 1 neonatal Sprague Dawley rats, plated at a cell density of 5,000 cells/well, and incubated in Biowhittaker Ultraculture containing 1% Penstrep, 1% L-glutamine, 0.7% ARAC, 3% rat serum, and NGF (50 ng/mL, Calomone Labs), at 37° C., under a 5% CO2 atmosphere. After 4 days the cells were treated with anti-NGF antibody (Sigma). At this time compound was added and the cells were maintained serum and NGF free for 48 hours, at which time viability of the neurons was assessed using Alamar Blue (Medicorp) staining. Table 3 summarizes selected IC₅₀ values from compounds tested using this protocol.

TABLE 3
Rescue from anti-NGF killing of cultured SCG neurons.

| Compounds | IC₅₀ (μM) | Compound | IC₅₀ (μM) | Compound | IC₅₀ (μM) |
|---|---|---|---|---|---|
| 5 | >30 | 29 | >30 | 48 | 28 |
| 6 | >30 | 30 | >30 | 49 | 40% at 50 μM |
| 12 | >30 | 31 | 35 | 50 | 22 |
| 13 | >30 | 32 | 30 | 51 | 38% at 30 μM |
| 14 | 30 | 33 | >30 | | |
| 15 | 40% at 10 μM | 34 | 24 | 53 | >30 |
| 16 | 42% at 30 μM | 35 | 40% at 30 μM | 54 | 23 |
| 17 | 25 | 36 | 16 | 55 | 7 |
| 18 | >30 | 37 | >30 | 56 | 40 |
| 20 | >50 | 38 | 40% at 50 μM | 57 | 50 |
| 21 | 50 | 39 | 30 | 58 | 50 |
| 22 | >30 | 41 | 7 | 59 | 38% at 50 μM |
| 23 | 25 | 42 | 17 | | |
| 24 | >30 | 43 | 7 | | |
| 25 | 17 | 44 | 20 | | |
| 26 | 16 | 45 | 7 | | |

### Example 63

In Vitro Protection of SCG neurons from Taxol killing. SCG neurons were isolated from day 1 neonatal Sprague Dawley rats, plated at a cell density of 10,000 cells/well, and incubated in Biowhittaker Ultraculture containing 1% Penstrep, 1% L-glutamine, 0.7% ARAC, 3% rat serum, and NGF (50 ng/mL, Calomone Labs) at 37° C., under a 5% CO₂ atmosphere. After 5 days the cells were treated with compound and Taxol™ (50 ng/mL). Viability of neurons was assessed 48 hours later using MTS (Promega) staining. Table 4 summarizes selected IC₅₀ values from compounds tested using this protocol. Also, see FIG. 1 for compound protection against Taxol killing.

TABLE 4
Rescue from Taxol killing of SCG neurons.

| Compounds | IC₅₀ (μM) | Compound | IC₅₀ (μM) |
|---|---|---|---|
| 1 | 25 | 22 | 10 |
| 2 | 50 | 25 | 38% at 30 μM |
| 3 | 10 | 26 | 7 |
| 4 | 8 | 55 | 2 |

### Example 64

In Vitro Protection of SCG neurons from cisplatin killing. SCG neurons were isolated from day 1 neonatal Sprague Dawley rats, plated at a cell density of 10,000 cells/well, and incubated in Biowhittaker Ultraculture containing 1% Penstrep, 1% L-glutamine, 0.7% ARAC, 3% rat serum, and NGF (50 ng/mL, Calomone Labs) at 37° C., under a 5% CO₂ atmosphere. After 5 days the cells were treated with compound and cisplatin (3 μg/mL). Viability was assessed 48 hours later using MTS (Promega) staining.

TABLE 5
Protection of SCG neurons against cisplatin killing

| Entry | Compound | IC₅₀ (±1 μM) |
|---|---|---|
| 1 | 23 | 15 |

### Example 65

Phosphatase Profile—SHP1, SHP2, PTP-1B, LAR, CD45

A) SHP1 and SHP2: SHP1 and SHP2 adenovirus vectors were prepared. SHP1 and SHP2 were independently over expressed, isolated and purified using FPLC. Enzyme was diluted to 1000-1400 FU/min. Enzyme (45 μL) was uniformLy plated in 96 well plates and treated with serial dilutions of compound (2 μL in DMSO). Compound and enzyme are allowed to incubate for 30 minutes at RT before the addition of DIFMUP (100 μM). The solutions are allowed to incubate at RT for 10 minutes prior to reading the fluorescence, to provide IC₅₀ curves using LSW Data Analysis Tool Box (MDL).

B) PTP-1B Assay: Inhibition of the phosphatase PTP1B was performed according to reported procedures (Puius, Y. A. et a. Proc. Natl. Acad. Sci., 1997, 94, 13420; Liu, F. J. Biol. Chem., 1996, 271, 31290). PTP1B was purchased from BIOMOL Research Laboratories, Inc.

C) LAR Assay: Inhibition of the phosphatase LAR was performed according to reported procedures (Cho, H. Biochemistry 1991, 30, 6210; 1992, 31, 133; Cho, H. Prot. Sci., 1993, 2, 977). LAR was purchased from BIOMOL Research Laboratories, Inc.

D) CD45 (PTPase) Assay: Inhibition of the phosphatase CD45 was performed according to reported procedures (Pacitti, A. et al Biochem. Biophys. Acta, 1994, 1222, 277; Fisher, D. K. and Higgins, T. J. Pharmacol. Res., 1994, 11, 759). CD45 was purchased from BIOMOL Research Laboratories, Inc.

E) PP1 Assay: Inhibition of the phosphatase PP1 was performed according to reported procedures (Nat. Biotechnol., 2000, 18, 847).

TABLE 6
Phosphatase Selectivity

| | SHP1 | SHP2 | PTP-1B | LAR | C45 | PP1 |
|---|---|---|---|---|---|---|
| | IC₅₀ | IC₅₀ | IC₅₀ | IC₅₀ | IC₅₀ | IC₅₀ |
| Compound | (μM) | (μM) | (μM) | (μM) | (μM) | (μM) |
| 4 | 42.7 | — | no inh. | no inh. | 62.2 | no inh. |
| 23 | 10.9 | 16.5 | no inh. | >100 | no inh. | no inh. |
| 30 | 5.41 | 18.4 | no inh. | no inh. | no inh. | no inh. |

### Example 66

TrkA Phosphorylation Assay: Culture media was removed from cultured PC12 cells (106-107, 50% confluent) and media containing NGF (1 to 50 ng/mL), 0.1% BSA, and compound (1 to 30 μM) was added. The cells were incubated for 1 to 60 minutes, the media was removed and the cells washed twice with ice cold TBS solution. Lysis buffer was added (1× TBS, 1% (v/v) NP-40, 10% (v/v) glycerol, 1 mM PMSF, 10 μg/mL leupeptin, and 0.5 mM sodium orthovanadate) and the cells were rocked for 20 minutes at 4° C. The cells were harvested and spun down (10,000 G for 10 min at 4° C.) and the supernatant was incubated with rabbit anti-Trk antibody (supplier) for 2 hours at 4° C. A 50% solution of protein A-Sepharose CL-4B or agarose was added and the mixture rocked for an additional 1-2 hours. The beads were spun down and washed 3 times with ice cold lysis buffer. Sample buffer (1×, 10% (v/v) glycerol, 2% (v/v) sodium dodecyl sulphate, 0.1M dithiothreitol, 0.005% bromophenol blue) is added and heated to 90° C. for 5 minutes. The sample was spun down and loaded onto a 7.5% SDS polyacrylamide gel (29:1 acrylamde:bis) and electrophoresed overnight at 50 volts. Alternatively, protein was transferred to nitrocellulose for 1 to 4 hours at 0.5 amps, 4° C., in transfer buffer, blocked for 1 hour with 2% BSA in TBS at room temperature, rinsed twice with TBS for 15 minutes, and incubated overnight at 4° C. in anti-phosphotyrosine.

The above-described embodiments of the present invention are intended to be examples only. Alterations, modifications and variations may be effected to the particular embodiments by those of skill in the art without departing from the scope of the invention, which is defined solely by the claims appended hereto.

1-27. (canceled)

1. A compound represented by Formula I 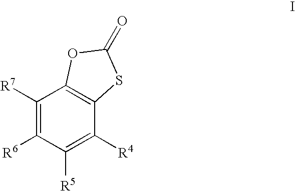
or pharmaceutically acceptable salts thereof wherein:
R⁴, R⁵, R⁶, and R⁷ are independently selected from the group consisting of:
H, halogen, cyano, azide, formyl, substituted and unsubstituted C(1-8)alkyl, C(1-8)fluoroalkyl, substituted and unsubstituted aralkyl, substituted and unsubstituted aryl, substituted and unsubstituted heteroaryl, substituted and unsubstituted biphenyl;
XR⁸, wherein X is S or O, and R⁸ is selected from the group consisting of H, substituted and unsubstituted C(1-8) alkyl, C(1-8)fluoroalkyl, substituted and unsubstituted acyl, substituted and unsubstituted arylcarbonyl, substituted and unsubstituted heteroarylcarbonyl, substituted and unsubstituted alkylaminocarbonyl, substituted and unsubstituted arylaminocarbonyl, substituted and unsubstituted heteroarylaminocarbonyl, substituted and unsubstituted aralkyl substituted and unsubstituted aryl, substituted and unsubstituted heteroaryl, substituted and unsubstituted alkylsulfonyl, substituted and unsubstituted arylsulfonyl, substituted and unsubstituted heteroarylsulfonyl; and
NR⁹R¹⁰, wherein R⁹ and R¹⁰ are independently selected from the group consisting of H, substituted and unsubstituted C(1-8)alkyl, C(1-8)fluoroalkyl,substituted and unsubstituted acyl, substituted and unsubstituted arylcarbonyl, substituted and unsubstituted heteroarylcarbonyl, substituted and unsubstituted alkylaminocarbonyl, substituted and unsubstituted arylaminocarbonyl, substituted and unsubstituted heteroarylaminocarbonyl, substituted and unsubstituted aralkyl, substituted and unsubstituted aryl, substituted and unsubstituted heteroaryl, substituted and unsubstituted alkylsulfonyl, substituted and unsubstituted arylsulfonyl, substituted and unsubstituted heteroarylsulfonyl, or wherein R⁹ and R¹⁰ are combined along with the N to which they are attached to form a heteroalkyl, substituted heteroalkyl, heteroaryl, and substituted heteroaryl ring system; and wherein
R⁴ and R⁵ may be combined to form a cycloalkyl, substituted cycloalkyl, heterocycloalkyl, substituted heterocycloalkyl, aryl, substituted aryl, heteroaryl, or substituted heteroaryl ring system; and
R⁶ and R⁷ may be combined to form a cycloalkyl, substituted cycloalkyl, heterocycloalkyl, substituted heterocycloalkyl, aryl, substituted aryl, heteroaryl, or substituted heteroaryl ring system,
with the proviso that the following compounds are excluded: 5-(N-cyclohexylcarbamoyloxy)-7-methylbenzo[1,3]oxathiol-2-one (31), 5-(3-chlorobenzothiophen-2-ylcarbonyloxy)-7-phenylbenzo[1,3]oxathiol-2-one (33), 6-(4-nitrophenylcarbonyloxy)-7-phenylbenzo[1,3]oxathiol-2-one (34), 5-hydroxy-7-(4-fluorophenyl)benzo[1,3]oxathiol-2-one (35), 5-hydroxy-7-(2-chlorophenyl)benzo[1,3]oxathiol-2-one (36), 5-hydroxy-7-(3-chlorophenyl)benzo[1,3]oxathiol-2-one (37), 5-(2-chlorophenylcarbonyloxy)-7-(3-chlorophenyl)benzo[1,3]oxathiol-2-one (38), 5-hydroxy-7-(4-chlorophenyl)benzo[1,3]oxathiol-2-one (39), 5-(2-chlorophenylcarbonyloxy)-7-(4-chlorophenyl)benzo[1,3]oxathiol-2-one (40), 5-hydroxy-7-(2,4-dichlorophenyl)benzo[1,3]oxathiol-2-one (41), 5-hydroxy-7-(2,5-dichlorophenyl)benzo[1,3]oxathiol-2-one (42), 5-hydroxy-7-(3,4-dichlorophenyl)benzo[1,3]oxathiol-2-one (43), 5-hydroxy-7-(4-bromophenyl)benzo[1,3]oxathiol-2-one (44), 5-hydroxy-7-(3-methylphenyl)benzo[1,3]oxathiol-2-one (46), 5-hydroxy-7-(4-methylphenyl)benzo[1,3]oxathiol-2-one (47), 5-(2-chlorophenylcarbonyloxy)-7-(3-methylphenyl)benzo[1,3]oxathiol-2-one (48), 5-hydroxy-7-(2-trifluoromethylphenyl)benzo[1,3]oxathiol-2-one (51), 5-hydroxy-7-(4-methoxyphenyl)benzo[1,3]oxathiol-2-one (53), 7-ethylamino-5-methylbenzo[1,3]oxathiol-2-one (56), 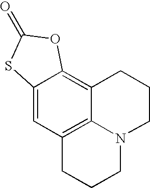
(57), 5-hydroxy-7-((2-naphthy)sulfanyl)benzo[1,3]oxathiol-2-one (23), 5-(N-Butylcarbamoyloxy)-7-((2-naphthyl)sulfanyl)benzo[1,3]oxathiol-2-one (25), and 4-hydroxy-3-((2-naphthyl)sulfanyl)naphtha[2,1-d]1,3-oxathiol-2-one (60),
naphtho[1,2-d]-1,3-oxathiol-2-one; 4,6-dimethyl-5-hydroxy-1,3-oxathiol-2-one, 6-hydroxy-1,3-benzoxathiol-2-one; 4,6-hydroxy-1,3-benzoxathiol-2-one,
7-hydroxy-1,3-benzoxathiol-2-one; 4-methyl-6-hydroxy-1,3-benzoxathiol-2-one, 5-chloro-6-hydroxy-1,3-benzoxathiol-2-one; 5-bromo-6-hydoxy-1,3-benzoxathiol-2-one, 6-hydroxy-7-methyl-1,3-benzoxathiol-2-one; 3-hydroxy-naphthyl-[1,2]1,3-benzoxathiol-2-one, 7-hydroxy-naphthyl-[1,2]1,3-benzoxathiol-2-one;
and with the further proviso that when R⁴ is H, R⁵ is OH, R⁶ is H, then R⁷ is not unsubstituted phenyl;
and with the further proviso that when R⁴ is H, R⁶ is H, R⁷ is H, then R⁵ is not methyl;
and with the further proviso that when R⁴ is H, R⁶ is H, R⁷ is H, then R⁵ is not OH;
and with the further proviso that when R⁴ is H, R⁵ is OH, R⁶ is H and R⁷ is substituted phenyl then the substituent is not 3-methyl, 4-methyl, 4-methoxy, 2-chloro, 3-chloro, 4-chloro, or 4-nitro;
and with the further proviso that when R⁴ is H, R⁵ is OH, R⁷ is H and R⁶ is substituted phenyl, then the substituent is not 3-methyl, 4-methyl, 4-methoxy, 2-chloro, 3-chloro, 4-chloro, or 4-nitro;
and with the further proviso that when R⁷ is H, R⁶ is H, R⁵ is OH and R⁴ is substituted phenyl, then the substituent is not 3-methyl, 4-methyl, 4-methoxy, 2-chloro, 3-chloro, 4-chloro, or 4-nitro;
and with the further proviso that when R⁴ is H, R⁶ is H, R⁷ is unsubstituted phenyl, then R⁵ is not OC(O)-phenyl;
and with the further proviso that when R⁴ is H, R⁶ is H, R⁷ is unsubstituted phenyl, R⁵ is substituted OC(O)-phenyl, then the phenyl substituent is not 4-nitro;
and with the further proviso that when R⁴ is H, R⁶ is H, R⁷ is unsubstituted phenyl, then R⁵ is not 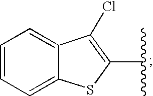
and with the further proviso that when R⁴ is H, R⁶ is H, R⁷is 3-methylphenyl, and R⁵ is substituted OC(O)-phenyl, then the phenyl substituent is not 2-chloro or 4-chloro;
and with the further proviso that when R⁴ is H, R⁶ is H, R⁷ is 4-methylphenyl, then R⁵ is not OC(O)-phenyl;
and with the further proviso that when R⁴ is H, R⁶ is H, R⁷ is 4-methylphenyl, and R⁵ is substituted OC(O)-phenyl, then the phenyl substituent is not 2-chloro or 4-chloro;
and with the further proviso that when R⁴ is H, R⁶ is H, R⁷ is 4-methoxyphenyl, then R⁵ is not OC(O)-phenyl;
and with the further proviso that when R⁴ is H, R⁶ is H, R⁷is 4-methoxyphenyl, and R⁵ is substituted OC(O)-phenyl, then the phenyl substituent is not 2-chloro or 4-chloro;
and with the further proviso that when R⁴ is H, R⁶ is H, R⁷is 2-chlorophenyl, then R⁵ is not OC(O)-phenyl;
and with the further proviso that when R⁴ is H, R⁶ is H, R⁷ is 2-chlorophenyl, and R⁵ is substituted OC(O)-phenyl, then the phenyl substituent is not 2-chloro, 4-chloro or 4-bromo;
and with the further proviso that when R⁴ is H, R⁶ is H, R⁷ is 4-chlorophenyl, then R⁵ is not OC(O)-phenyl;
and with the further proviso that when R⁴ is H, R⁶ is H, R⁷ is 2-chlorophenyl, and R⁵ is substituted OC(O)-phenyl, then the phenyl substituent is not 2-chloro, 4-chloro or 4-bromo;
and with the further proviso that when R⁴ is H, R⁶ is H, R⁷ is H, then R⁵ is not OC(O)NHCH₂C(O)OCH₂CH₃;
and with the further proviso that when R⁴ is H, R⁶ is H, R⁷ is H, then R⁵ is not OH;
and with the further proviso that when R⁴ is H, R⁶ is H, R⁷ is H, then R⁵ is not OC(O)NH-lower alkyl, OC(O)NH-substituted aryl, OC(O)NH-haloalkyl, OC(O)NH-carbalkoxyalkylene, or OC(O)NH-alkyl carbonyloxyalkylene;
and with the further proviso that when R⁴ or R⁵ is OH, and R⁶ is H, then R⁷ is not OH;
and with the further proviso that when R⁴ or R⁵ is OH, and R⁷ is H, then R⁶ is not OH;
and with the further proviso that when R⁴ or R⁵ is OH, and R⁶ is H, then R⁷ is not nitro;
and with the further proviso that when R⁴ or R⁵ is OH, and R⁷ is H, then R⁶ is not nitro;
and with the further proviso that when R⁴ or R⁵ is H, and R⁶ is H, then R⁷ is not OH;
and with the further proviso that when R⁴ or R⁵ is H, and R⁷ is H, then R⁶ is not OH;
and with the further proviso that when R⁴ or R⁵ is H, and R⁶ is H, then R⁷ is not nitro;
and with the further proviso that when R⁴ or R⁵ is H, and R⁷ is H, then R⁶ is not nitro.

2. The compound according to claim 1, wherein R⁷ is selected from the group consisting of substituted or unsubstituted arylthio, substituted or unsubstituted heteroarylthio, and R⁵ is selected from the group consisting of hydroxyl, substituted alkylcarbonyloxy or substituted alkylaminocarbonyloxy moiety containing an amino, mono- or disubstituted amino, pyridyl, piperidinyl, piperazinyl, morpholino, thiomorpholino, or pyrrolidinyl moiety, substituted or unsubstituted prolinyloxy, substituted or unsubstituted heteroarylcarbonyloxy, substituted or unsubstituted heteroarylaminocarbonyloxy.

3. The compound according to claim 1, wherein R⁷ is substituted or unsubstituted haloaryl, and R⁵ is selected from the group consisting of hydroxyl, substituted alkylcarbonyloxy or substituted alkylaminocarbonyloxy moiety containing an amino, mono- or disubstituted amino, pyridyl, piperidinyl, piperazinyl, morpholino, thiomorpholino, or pyrrolidinyl moiety, substituted or unsubstituted prolinyloxy, substituted or unsubstituted heteroarylcarbonyloxy, substituted or unsubstituted heteroarylaminocarbonyloxy.

4. The compound according to claim 1, wherein R⁵ is a substituted alkylcarbonyloxy or substituted alkylaminocarbonyloxy moiety containing an amino, mono- or disubstituted amino, pyridyl, or piperidinyl, piperazinyl, morpholino, or pyrrolidinyl moiety, substituted or unsubstituted heteroarylcarbonyloxy, substituted or unsubstituted heteroarylaminocarbonyloxy, substituted or unsubstituted prolinyloxy, and R⁷ is a substituted or unsubstituted biphenyl moiety.

5. The compound according to any one of claims 1 to 4, wherein R⁴ and R⁶ are hydrogen.

6. A compound, according to claim 1, selected from the group consisting of:

| Cpd | R⁴ | R⁵ | R⁶ | R⁷ |
|---|---|---|---|---|
| 1 | H | OH | H | H |
| 2 | H | 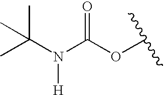 | H | H |
| 3 | Cl | OH | H | Cl |
| 4 | Cl | 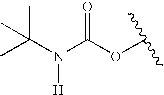 | H | Cl |
| 5 | H | OH | H | —SCH₂CH₃ |
| 6 | H | OH | H | —S(O)CH₂Ph |
| 7 | H | OH | H | —SCH₂CH₂Ph |
| 8 | H | OH | H | —SPh |
| 9 | H | OH | H | 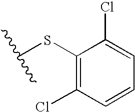 |
| 10 | H | OH | 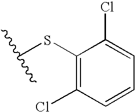 | H |
| 11 | H | OH | H | 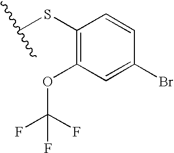 |
| 12 | H | OH | H | 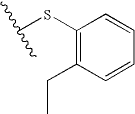 |
| 13 | H | OH | H | 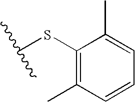 |
| 14 | H | OH | H | 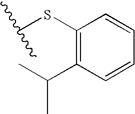 |
| 15 | H | OH | 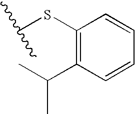 | H |
| 16 | H | OH | H | 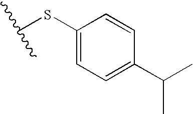 |
| 17 | H | OH | H | 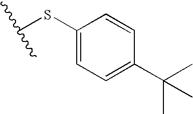 |
| 18 | H | OH | H | 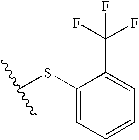 |
| 19 | H | OH | H | 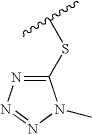 |
| 20 | 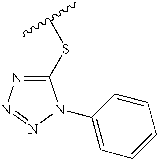 | OH | H | H |
| 21 | 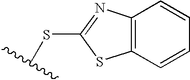 | OH | H | H |
| 22 | H | OH | H | 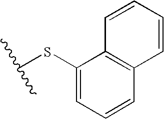 |
| 24 | H | OH | H | 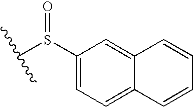 |
| 26 | H | 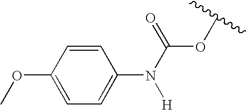 | H | 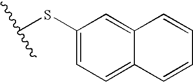 |
| 29 | 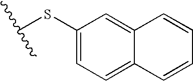 | OH | H | PhS— |
| 30 | 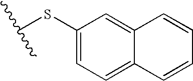 | OH | H | 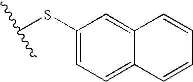 |
| 32 | H | OH | H | —Ph |
| 45 | H | OH | H | 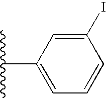 |
| 49 | H | OH | H | 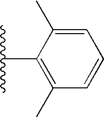 |
| 50 | H | 0H | 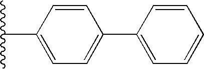 | H |
| 54 | H | OH | H | 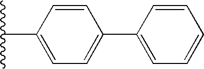 |
| 55 | H | 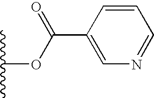 | H | 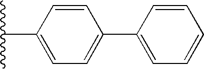 |
| 58 | H | 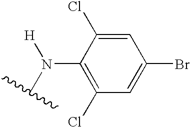 | H | H |
| 59 | H | 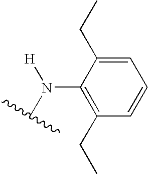 | H | H |
| 61 | H | 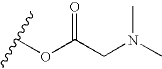 | H | 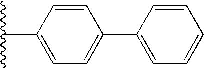 |

7. A composition for the prevention of neuronal cell loss or for the treatment of nerve cell or axonal degradation, comprising a compound, according to claim 1, but without the provisos, together with a suitable pharmaceutically acceptable diluent or carrier.

8. A method for the prevention of neuronal cell loss or for the treatment of nerve cell or axonal degradation, the method comprising: administering to a subject in need thereof an effective amount of the composition, according to claim 7, so as to prevent the neuronal cell loss or to treat the nerve cell or axonal degradation.

9. A method for the prevention or treatment of a neurodegenerative disease of the central and/or peripheral nervous systems, the method comprising: administering to a subject in need thereof an amount of the composition, according to claim 7, so as to prevent or treat the neurodegenerative disease.

10. The method, according to claim 9, in which the neurodegenerative disease is neurodegenerative diseases of the eye.

11. The method, according to claim 10, in which the neurodegenerative disease of the eye is macular degeneration and glaucoma.

12. The method, according to claim 9, in which the neurodegenerative disease is Alzheimer's disease, Parkinson's disease, or amyotrophic lateral sclerosis (ALS).

13. A method of treating axonal degradation, the method comprising; administering to a subject in need thereof an effective amount of the composition, according to claim 7, so as to treat the axonal degradation.

14. Use of the compound, according to claim 1, without the provisos, for altering signal transduction.

15. A method of inducing axonal growth, the method comprising; administering to a subject in need thereof an effective amount of the composition, according to claim 7, so as to induce axonal growth.

16. A method of treating or preventing neuropathies and neuropathic pain in a subject, the neuropathies and neuropathic pain resulting from axonal and/or neuronal cell body damage, and/or from the loss of axonal growth and repair, the method comprising administering to the subject an effective amount of the composition, according to claim 7.

17. The method, according to claim 16, in which the neuropathy is peripheral neuropathy

18. The method, according to claim 17, in which the neuropathy results from a toxic agent.

19. The method, according to claim 18 in which the toxic agent is a neurotoxic agent selected from Table 1.

20. The method, according to claim 19, in which the neurotoxic agent is a chemotherapeutic agent.

21. A method of treating a neurodegenerative disease, the method comprising: coadministering to a subject in need thereof, an effective amount of the composition, according to claim 7, in combination with other compounds known to be useful to treat the neurodegenerative disease.

22. The method, according to claim 21, in which the other compounds include acetylcholinesterase inhibitors, L-dopa, angiotensin-converting enzyme inhibitors (ACE inhibitors) or insulin.
